(19) United States
(12) Patent Application Publication
(10) Pub. No.: US 2024/0206920 A1
(43) Pub. Date: Jun. 27, 2024

(54) SYSTEM AND METHOD FOR LUMBAR FUSION

(71) Applicant: PROVIDENCE MEDICAL TECHNOLOGY, INC., Pleasanton, CA (US)

(72) Inventors: Christopher U. Phan, Dublin, CA (US); Nicholas Domek, Sacramento, CA (US)

(21) Appl. No.: 18/288,641

(22) PCT Filed: Apr. 28, 2022
(86) PCT No.: PCT/US2022/026638

§ 371 Date: Oct. 27, 2023

Related U.S. Application Data
(60) Provisional application No. 63/181,507, filed on Apr. 29, 2021.

(51) Int. Cl. A61B 17/70; A61B 17/16

(52) U.S. Cl. CPC A61B 17/7064; A61B 17/1604; A61B 17/1657; A61B 17/1659; A61B 17/1671

(57) ABSTRACT

A lumbar facet cage or implant and a system and method for delivery of the implant to the lumbar spine to immobilize the spine and promote fusion are described. In one aspect, the system and method are indicated for the fixation of the spine as an aid to fusion through immobilization of the lumbar facet joint. In one aspect, a cage or implant sized for the lumbar facet joint space that fixates the superior articular and inferior articular facets of the treated facet joint is described. In one aspect, the cage or implant includes one or more stop features.

### CROSS REFERENCE TO RELATED APPLICATIONS

This application claims the benefit of priority to U.S. Patent Application No. 63/181,507, filed Apr. 29, 2021 and titled “System and Method for Lumbar Fusion” and which is hereby incorporated by reference in its entirety.

### BACKGROUND

Spinal fusion is a surgical technique that joins two or more vertebrae by promoting bone growth and ultimately hindering or preventing movement between fused vertebrae. It can be performed at any level of the spine (lumbar, thoracic and cervical) and is often performed to alleviate pain from disc degeneration, spinal stenosis and other spinal column injuries or deformities. Improved systems and methods for fusion, particularly lumbar fusion, are needed.

### SUMMARY

A lumbar facet implant or cage and a system and method for delivery of the implant to the lumbar spine to immobilize the spine and promote fusion are described. In one aspect, the system and method are indicated for the fixation of the spine as an aid to fusion through immobilization of the lumbar facet joint. In one aspect, an implant sized for the lumbar facet joint space that fixates the superior articular and inferior articular facets of the treated facet joint is described. In one aspect, the implant includes one or more stop features, and is configured to receive at least two bone screws that anchor into the articular processes of the treated level for further securement and fixation of the implant to the facet joint. Bone graft may be added to the implant open space and surrounding areas to encourage facet fusion.

A lumbar facet implant is described. In one aspect, the implant includes a proximal portion and a distal portion, a first surface opposite a second surface, the first and second surface each including one or more teeth, each of the first and second surfaces extending between the proximal and distal portions, and a stop feature extending from the proximal portion and generally perpendicular to one of the first surface or the second surface. The stop feature includes at least one opening configured to receive a fixation device.

In some aspects, the distal portion comprises a blunt distal tip. In some aspects, the stop feature includes rounded edges or corners. In some aspects, the stop feature is a semi-circle in shape. In some aspects, the stop feature increases a height but not a width of the implant. In some aspects, the stop feature extends from the first surface and includes two openings configured to receive a fixation device and the two openings are angled to extend above or away from the first surface. In some aspects, the stop feature includes three openings configured to receive a fixation device and two openings are angled to extend above or away from the first surface and one opening is angled to extend below or away from the second surface.

In some aspects, the implant further includes a second stop feature extending from the proximal portion and generally perpendicular to the other of the first or second surface. The first and second stop features may include rounded edges or corners. In some aspects, the first and second stop features increase a height but not a width of the implant. In some aspects, at least one of the first surface or the second surface comprises two rows of teeth. In one aspect, at least one of the first surface and the second surface comprises three rows of teeth. In some aspects, at least one of the first surface and the second surface comprises two openings or windows configured to receive bone graft material.

In some aspects, the fixation device is a screw.

A lumbar facet implant delivery system is described. The system includes a lumbar facet implant. In one aspect, the implant includes a proximal portion and a distal portion, the proximal portion including a release driver opening and a fixation device opening. The system also includes a delivery instrument. In one aspect, the delivery instrument includes an elongated body having a distal end and a proximal end, the distal end defining a lumbar facet implant receiving feature that is complimentary in shape to the proximal portion of the lumbar facet implant. The system may further include a release driver configured to engage the release driver opening of the proximal portion of the implant. The system may further include a tubular body defined within the elongated body that is configured to receive the fixation device and change the trajectory of the fixation device being delivered to the fixation device opening of the implant.

The lumbar facet implant may be an implant as described herein.

A method of delivering a lumbar facet implant to a lumbar facet space is described. In one aspect, the method includes introducing a guide portal to the lumbar facet space. In some aspects, the method may further include introducing a lumbar facet implant delivery system with a lumbar facet implant through the guide portal and securing the lumbar facet implant in the lumbar facet space. In some aspects, the method may further include accessing the lumbar facet space with an access chisel. The lumbar facet space may be decorticated and the access chisel may be removed through the guide portal.

The lumbar facet implant used with the method may be an implant as described herein.

In some aspects, the lumbar facet space is any facet joint at L1-S1.

A system for lumbar fusion is disclosed. The system includes an access chisel, at least one decorticator, and a guide tube.

In some aspects, the access chisel comprises a proximal end opposite a distal portion, the distal portion having a thin, forked, tapered tip, a positive stop and an arcuate shaped cut out opposite the positive stop.

In some aspects, the at least one decorticator is a first decorticator comprises a tubular body and a proximal portion opposite a distal portion, the distal portion including bidirectional teeth in a 180° degree array or 360° degree array.

In some aspects, the first decorticator has the bidirectional teeth in a 180° array and further includes an arcuate cut out feature opposite the bidirectional teeth.

In some aspects, the first decorticator is adapted to slide over the access chisel.

In some aspects, the at least one decorticator is a second decorticator comprising a tubular body and a proximal portion opposite a distal portion, the distal portion including bidirectional teeth in a 180° degree array, a 360° degree array or a 360° degree array with a center countersink.

In some aspects, the second decorticator has the bidirectional teeth in a 180° array and further includes a linear or straight edge cut out feature opposite the bidirectional teeth.

In some aspects, the second decorticator is received in the guide tube.

In some aspects, the at least one decorticator is a third decorticator comprising a solid tubular body and a proximal portion opposite a distal portion, the distal portion including one side having a keel and a second side having a rasp.

In some aspects, the third decorticator is received in the guide tube.

In some aspects, the at least one decorticator comprises a first decorticator, a second decorticator and a third decorticator.

In some aspects, the guide tube comprises a tubular body and a proximal portion opposite a distal portion, the distal portion including a pair of arms, a positive stop and an arcuate shaped cut out opposite the positive stop.

In some aspects, the system includes the lumbar facet implant delivery system described herein.

Additional embodiments and features are set forth in part in the description that follows, and will become apparent to those skilled in the art upon examination of the specification of may be learned by the practice of the disclosed subject matter. A further understanding of the nature and advantages of the present disclosure may be realized by reference to the remaining portions of the specification and the drawings, which form a part of this disclosure. One of skill in the art will understand that each of the various aspects and features of the disclosure may advantageously be used separately in some instances, or in combination with other aspects and features of the disclosure in other instances.

### BRIEF DESCRIPTION OF THE DRAWINGS

The description will be more fully understood with reference to the following figures in which components are not drawn to scale, which are presented as various examples of the present disclosure and should not be construed as a complete recitation of the scope of the disclosure, characterized in that:

FIGS. 1A to 2H-3 depict various embodiments of a lumbar facet implant.

FIGS. 3A-1 to 3D depict a lumbar facet implant (such as an implant shown in FIGS. 1A-2H-3) and implant delivery device.

FIGS. 4A-1 to 4A-3 depict various views of a facet access device.

FIGS. 4B-1 to 4B-6 illustrate various views and embodiments of a first or outer decorticator.

FIGS. 4C-1 to 4C-4 illustrate various views of a lumbar access portal or guide tube.

FIGS. 4D-1 to 4D-6 illustrate various views and embodiments of a second or intra-portal decorticator.

FIGS. 4E-1 to 4E-5 illustrate various views of a third or intra-facet decorticator.

FIGS. 5A-1 to 5A-5 depict the facet access device in use in accordance with the systems and methods described herein.

FIGS. 5B-1 to 5B-6 depict the first or outer decorticator in use in accordance with the systems and methods described herein.

FIGS. 5C-1 to 5C-6 depict the lumbar access portal or guide tube in use in accordance with the systems and methods described herein.

FIGS. 5D-1 and 5D-2 depict the second or intra-portal decorticator in use in accordance with the systems and methods described herein.

FIGS. 5E-1 to 5E-4 depict the third or intra-facet decorticator in use in accordance with the systems and methods described herein.

FIGS. 5F-1 to 5F-5 depict the lumbar facet implant and implant delivery device in use in accordance with the systems and methods described herein.

FIGS. 5G-1 to 5G-5 depict a bone graft and bone tamp in use in accordance with the systems and methods described herein.

FIG. 6 describes a method for lumbar fusion.

### DETAILED DESCRIPTION

Disclosed herein are various devices, systems and methods for use in fusion of the lumbar spine. A lumbar facet cage or implant is sized for the lumbar facet joint space and fixates the superior articular and inferior articular facets of the treated facet joint. The cage or implant accepts a fixation device, such as a screw, to anchor into the articular processes of the treated level for further securement and fixation of the cage to the facet joint. Bone graft may then be applied to the open space within the cage and surrounding areas to promote facet fusion.

A system for lumbar facet fusion may include various instruments. The system may include an access instrument or access chisel to locate and anchor into the lumbar facet joint. A first or outer decortication instrument is used to restructure the posterior facet surface for the access portal or guide tube and to prepare the joint for fusion. The access portal or guide tube is anchored into the lumbar facet joint and provides a working cannula for subsequent instruments. A second or intra-portal decortication instrument further prepares the posterior facet surface for fusion. A third or intra-facet decortication instrument is used to decorticate and remove tissue from the articular surfaces of the facet joint. A bone tamp instrument may be used to allograft or autograft to the facet joint.

The method for lumbar fusion may use the above described instruments. Posterior access to the lumbar spine may be used in the method. The levels for treatment may be at any of L1-S1 and may include multiple levels. The method may be performed bilaterally or only on one side at any given level. In one example of the method, the facet joint is accessed, with a chisel like instrument, from a posterior approach. Then, decortication of the facet edge with a trephine that slides over the access chisel is performed. Then, the guide portal is inserted over the access chisel and the access chisel is removed. Further decortication of the facet joint is performed and remodeling of bone in surrounding areas through the guide portal in preparation of cage implantation is performed. The implant is then introduced into the lumbar facet joint through the guide portal. The screws are placed into the implant via the cage delivery instrument and bone graft is delivered into the surgical space through the guide portal and tamped down into the surrounding area.

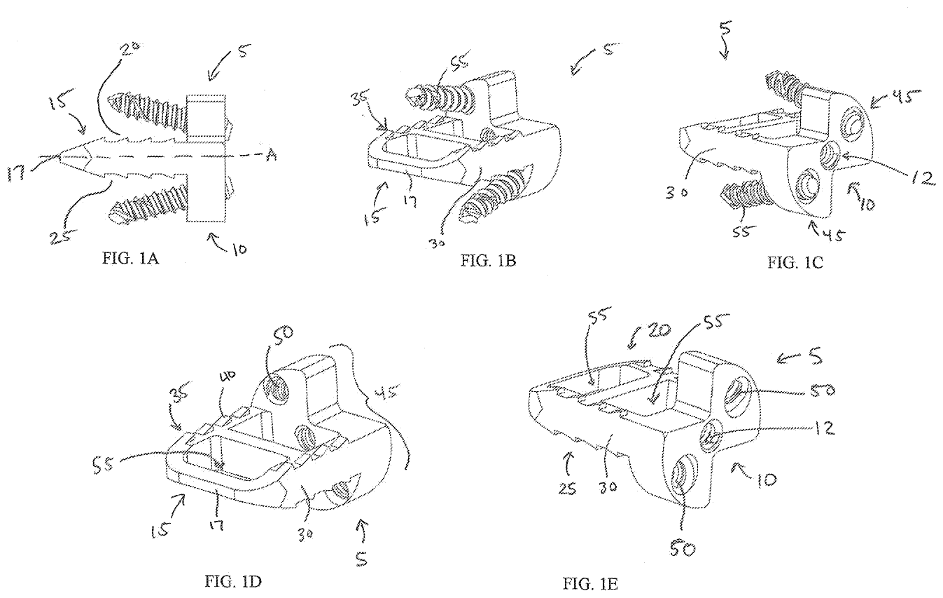

Turning now to the lumbar facet cage, FIGS. 1A-1C illustrate various views of a lumbar facet cage or implant and FIGS. 1D-1E depict the implant without the fixation members (e.g. screws). As can be understood, the cage or implant 5 includes proximal portion 10 and a distal portion 15, a top surface 20 and a bottom surface 25 and side surfaces 30, 35. The top and/or bottom surfaces 20, 25 may be flat or planar or may be curved or arcuate, or some combination. The top and/or bottom surfaces 20, 25 may also include bone engagement features 40, such as teeth. The teeth may be individual teeth aligned in single or multiple rows. The top and bottom surfaces include teeth to increase fixation in the facet joint space and resist facet motion.

The distal portion 15 may include a blunt distal tip 17 but the tip 17 may also be rounded or arcuate. The proximal portion 10 includes a threaded opening 12 to receive a delivery instrument attachment. The proximal portion 10 may include a stop feature 45 that includes tabs or “wings” that create a stop to prevent over insertion into the facet joint space. The tabs or wings, also referred to as a stop feature or features 45, may extend from the top or bottom surface, preferably perpendicular from the top or bottom surface, and generally have rounded or arcuate edges to aid in insertion through the delivery device. In some embodiments, the stop 45 may have three sides, where two sides are generally straight and the third side is generally arcuate or rounded. In the embodiment of FIGS. 1A-1E, there are two stops 45, one extending from the top surface and one extending from the bottom surface. In some aspects, the stop features 45 increase a height but not a width of the implant. That is, the stop feature 45 extends above and/or below a planar or horizontal surface of the implant 5 to increase the height of the implant 5 but the stop feature 45 does not extend parallel to or laterally from the planar surface of the implant and therefore does not increase the width of the implant. The tabs or wings or stop feature(s) also include openings 50, which may be offset from and angled relative to the threaded opening 12 for the delivery instrument, and the openings 50 receive screws or other fixation devices 55 to anchor the cage 5 to the articular process of the respective vertebra. The openings 50 are at an angle to the horizontal axis A running centrally through the cage. The top and bottom surfaces 20, 25 include openings or windows 55 in the cage or implant that receive bone graft material to aid in or promote fusion.

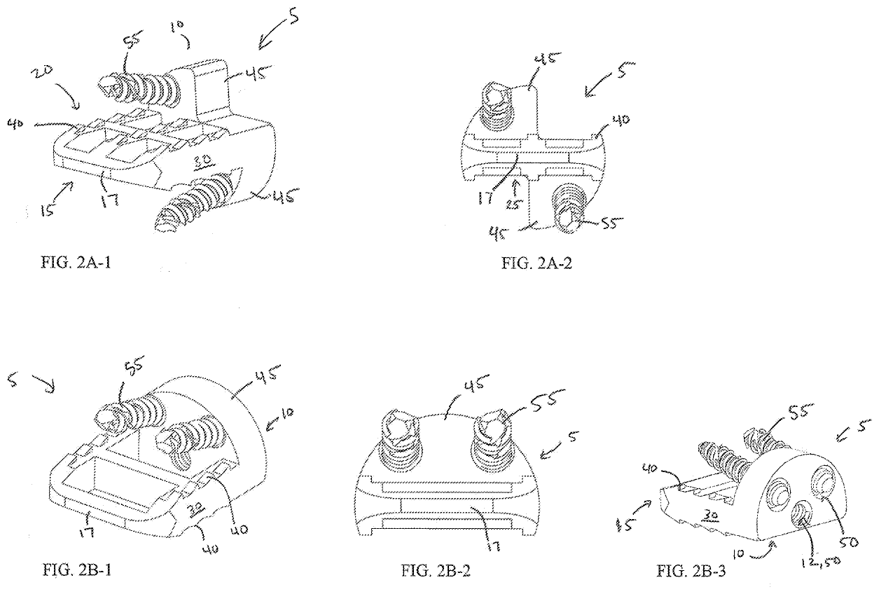

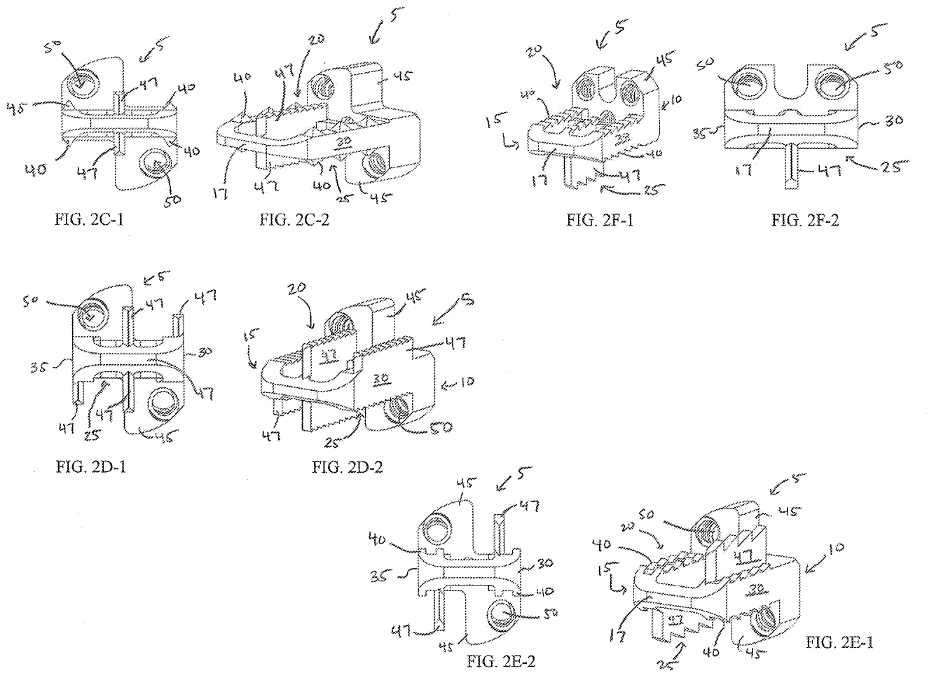

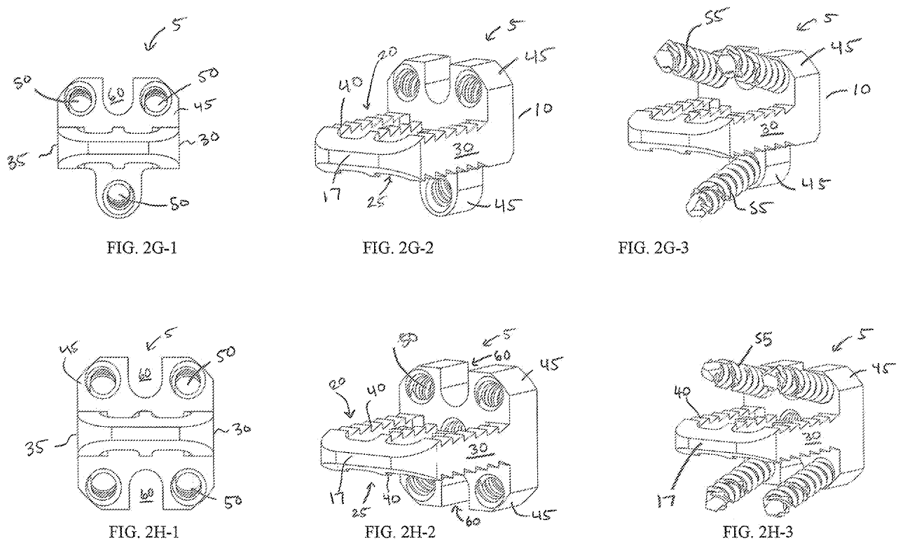

FIGS. 2A-1 and 2A-2 depict another embodiment of the cage or implant 5. In this embodiment, the cage or implant 5 includes many of the same features of FIG. 1A-1E as described above, but also includes three rows of teeth 40 on the top and bottom surface 20, 25. FIGS. 2B-1 through 2B-3 depict another embodiment of the implant or cage 5 and include many of the same features. However, this implant 5 includes a stop feature 45 that is a half moon or semi-circular shape and includes two or optionally three openings 50 for a fixation device (e.g. screws 55). In embodiments where the third opening is used, the optional third opening may be angled in a direction that is opposite to the angle of the first two openings, e.g., two openings are angled such that the screw exits the implant through a top surface of the implant and the third optional opening 50 is angled such that a screw would exit the implant through the bottom surface of the implant. Alternatively, the third opening 12 may receive a portion of the implant delivery device.

FIGS. 2C-1 through 2E-2 depict various embodiments of the cage or implant 5 having many of the same features of FIG. 1A-1E as described above but also include second engagement features 47, such as a keel or projection 47. In some embodiments, there are no teeth 40 and the keel 47 may be the only engagement feature. The keels 47 channel into the bone above and below the joint space for added fixation. As can be understood from FIGS. 2C-1 to 2C-2, the keel 47 may extend centrally through the top and bottom surfaces 20, 25 of the cage or implant 5. In this example, the height of the keel 47 is greater than the height of the teeth 40. As can be understood from FIGS. 2D-1 to 2D-2, the keel 47 may extend centrally through the top and bottom surfaces 20, 25 of the cage or implant 5. In addition, a keel 47 may extend from the top surface 20 at a location proximate a side surface 30. Further, a keel 47 may extend from the bottom surface 25 at a location proximate the other side surface 35. The central keel 47 may have a height that is greater than either of the keels proximate a side surface. As can be understood from FIGS. 2E-1 to 2E-2, the keel 47 may extend from the top surface 20 at a location proximate to but not flush with (in the same lateral plane as) a side surface 30. In this example, a single row of teeth 40 extend from the top surface and are flush with (in the same lateral plane with) side surface 30. Proximate the other side surface 35 and extending from the top surface 20 are two rows of teeth 40. Further, a keel 47 may extend from the bottom surface 25 at a location proximate to but not flush with (in the same lateral plane as) the other side surface 35. In this example, a single row of teeth 40 extend from the bottom surface and are flush with (in the same lateral plane with) side surface 35. Proximate the other side surface 30 and extending from the bottom surface 25 are two rows of teeth 40. The keel 47 may have a height that is greater than any of the rows of teeth. As can be understood from FIGS. 2F-1 to 2F-2, the keel 47 may extend centrally from the bottom surface 25 of the cage or implant 5. In addition, teeth 40 may extend from the opposite end of the keel centrally through the top surface 20. Teeth 40 may also extend from the top and bottom surfaces and proximate to and flush with (in the same lateral plane as) the side surfaces 30, 35.

FIGS. 2G-1 through 2H-3 illustrate two other embodiments of the cage or implant 5 having many of the same features of FIG. 1A-1E as described above. As can be understood from FIGS. 2G-1 to 2G-3, the proximal portion 10 may include a stop feature 45 that includes tabs or “wings” that create a stop to prevent over insertion into the facet joint space. The tabs or wings, also referred to as a stop feature or features 45, may extend from the top or bottom surface, preferably perpendicular from the top or bottom surface, and generally have rounded or arcuate edges to aid in insertion through the delivery device. In the embodiment of FIGS. 2G-1 to 2G-3, there are three stops 45, two 45 extending from the top surface 20 and one extending centrally from the bottom surface 25. The two stops extending from the top surface are separated by a recess 60, such as a U-shaped recess. The tabs or wings or stop feature(s) also include openings 50 that receive screws 55.

As can be understood from FIGS. 2H-1 to 2H-3, the proximal portion 10 includes a threaded opening 12 to receive a delivery instrument attachment. The proximal portion 10 may include a stop feature 45 that includes tabs or “wings” that create a stop to prevent over insertion into the facet joint space. The tabs or wings, also referred to as a stop feature or features 45, may extend from the top or bottom surface, preferably perpendicular from the top or bottom surface, and generally have rounded or arcuate edges to aid in insertion through the delivery device. In the embodiment of FIGS. 2H-1 to 2H-3, there are four stops 45, two extending from the top surface and two extending from the bottom surface. The two stops 45 extending from the top surface 20 and the two stops 45 extending from the bottom surface 25 are each separated by a recess 60, such as a U-shaped recess. The tabs or wings or stop feature(s) also include openings 50 that receive screws 55.

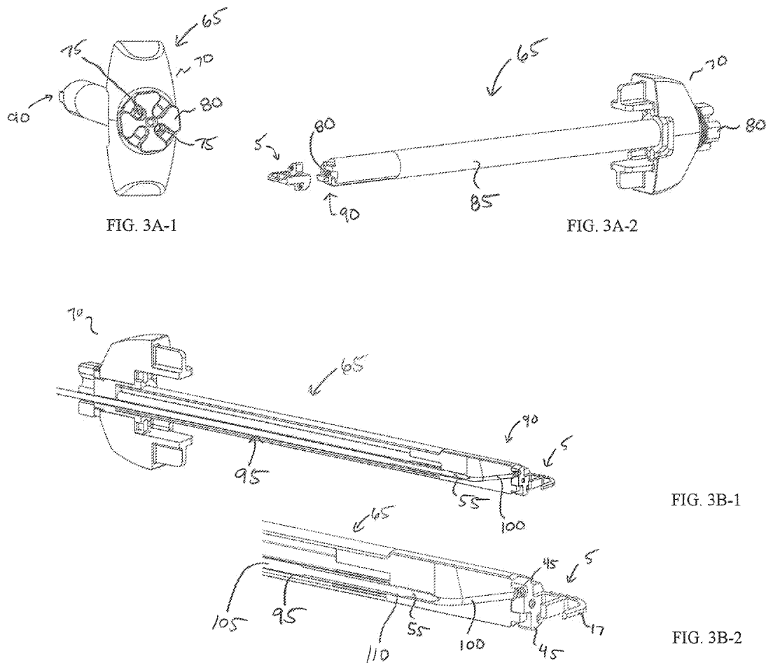

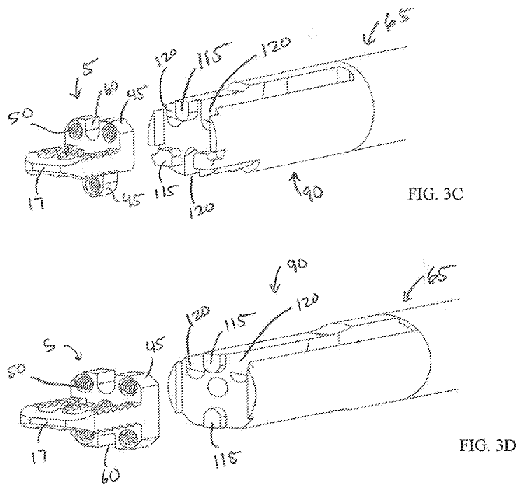

FIGS. 3A-1 to 3D depict a lumbar facet cage or implant 5 and implant delivery device or instrument 65. As shown in FIG. 3A-1, a proximal portion 70 of the delivery device 65 includes at least one screw portal or opening 75, preferably two screw portals or openings 75, to guide a screw to the implant. The screws are delivered to the implant while the implant is secured to the delivery instrument with a release driver 80. As shown in FIG. 3A-2, the release driver 80 extends through the body 85 of the delivery device 65 and is threadably engaged (to attach and detach from the implant) to the implant 5 where it exits from the distal end 90 of the delivery instrument. FIG. 3B-1 is a cross section of the delivery device 65 with an implant 5 attached and FIG. 3B-2 is an enlarged view of the distal portion 90 of the device 65 and the implant 5 of FIG. 3B-1. As can be understood from these figures, the delivery instrument 65 includes a tube 95, such as a hypotube, extending through the body 85 of the instrument and a ramp 100 that directs the screw 55 (coupled to a flexible shaft 105) out of the delivery instrument distal end 90 and into the implant 5. Between the screw 55 and the flexible shaft 105, there is a notch feature 110 that is torqued and when the screw is anchored into the implant and respective vertebra, the screw will break off from the shaft at the notch.

FIG. 3C illustrates a delivery device or instrument 65 that includes many of the same features of the delivery device described above except it may be used specifically with the implant of FIGS. 2G-1 to 2G-3. In this embodiment, the distal portion 90 of the delivery instrument 65 includes raised engagement features 115 to engage with the recess 60 of the implant 5 and/or the space next to the stop 45. Further, the delivery device 65 may include screw exit locations 120 that align with the threaded screw openings 50 in the respective stop features 45 of the implant 5.

FIG. 3D illustrates a delivery device or instrument 65 that includes many of the same features of the delivery device described above except it may be used specifically with the implant of FIGS. 2H-1 to 2H-3. In this embodiment, the distal portion 90 of the delivery instrument 65 includes raised engagement features 115 to engage with the recesses 60 of the implant 5. Further, the delivery device 65 may include screw exit locations 120 that align with the threaded screw openings 50 in the respective stop features 45 of the implant 5. In this embodiment, after a first set of screws are deployed, the delivery instrument is rotated to allow the second set of screws to be deployed.

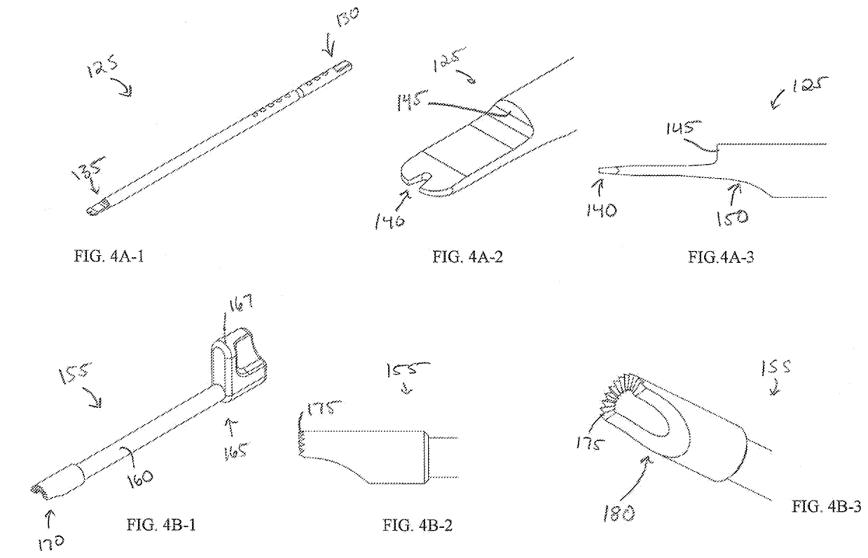

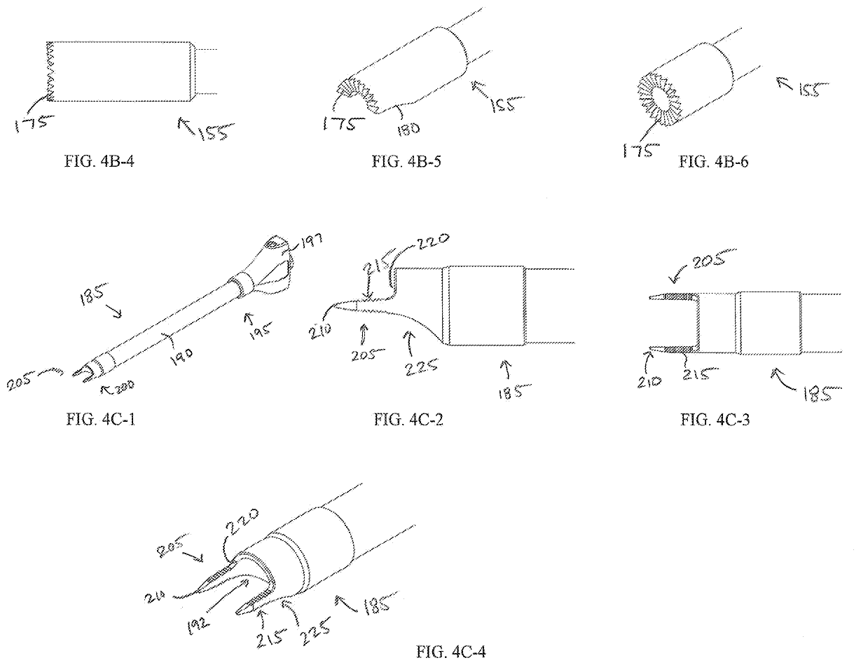

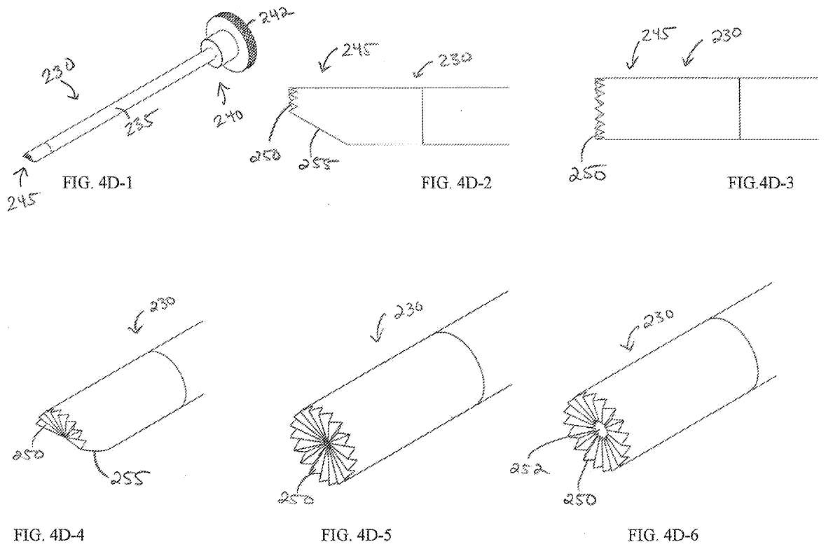

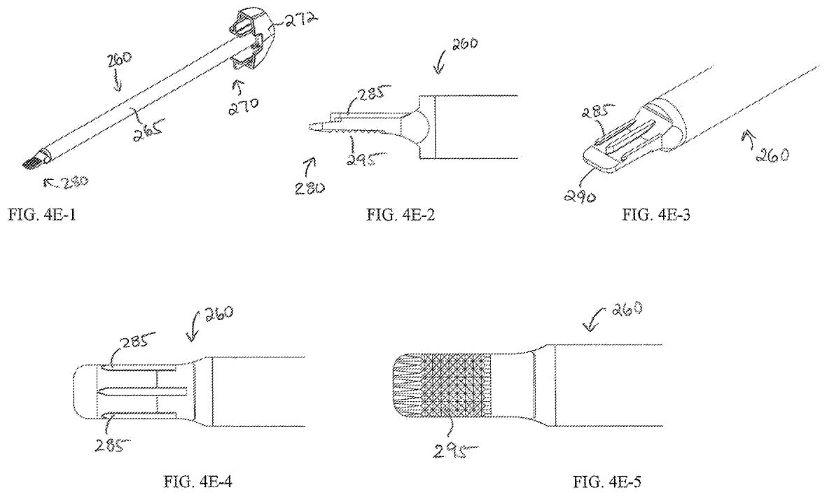

Turning now to FIGS. 4A-1 through FIGS. 4E-5, a system or set of instruments may be used to perform lumbar facet fusion from a posterior approach. In some embodiments, the system or set of instruments may include the implant and implant delivery device described previously. FIGS. 4A-1 through FIG. 4A-3 depict various views of an access instrument or access chisel 125. The access instrument 125 is used to locate and anchor into the lumbar facet joint. As shown in the isometric view of FIG. 4A-1, the access chisel 125 has a generally cylindrical body with a proximal end 130 adapted to receive a handle (not shown) and a distal portion 135 adapted for insertion in the joint. FIGS. 4A-2 and 4A-3 are enlarged views of the distal portion 135 including a thin, forked and tapered tip 140 to stabilize the chisel upon entrance into the joint and to help penetrate the facet capsule prior to entry into the joint. The distal portion 135 further includes a positive stop 145 to abut bone on the posterior edge of the superior or inferior facet surface to prevent over insertion. The distal portion further includes an arcuate shaped cutout 150 opposite the positive stop 145 that may prevent tissue interference during insertion.

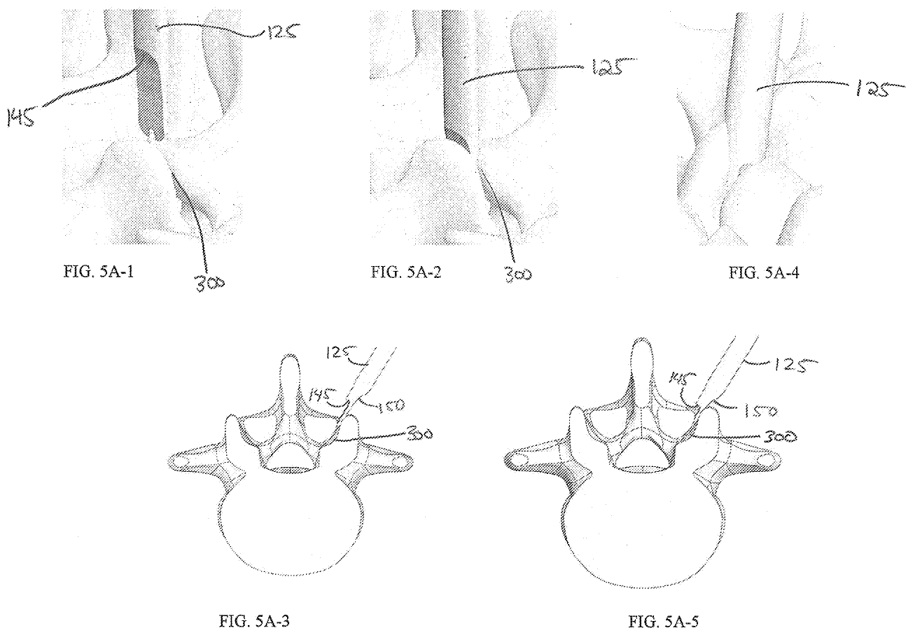

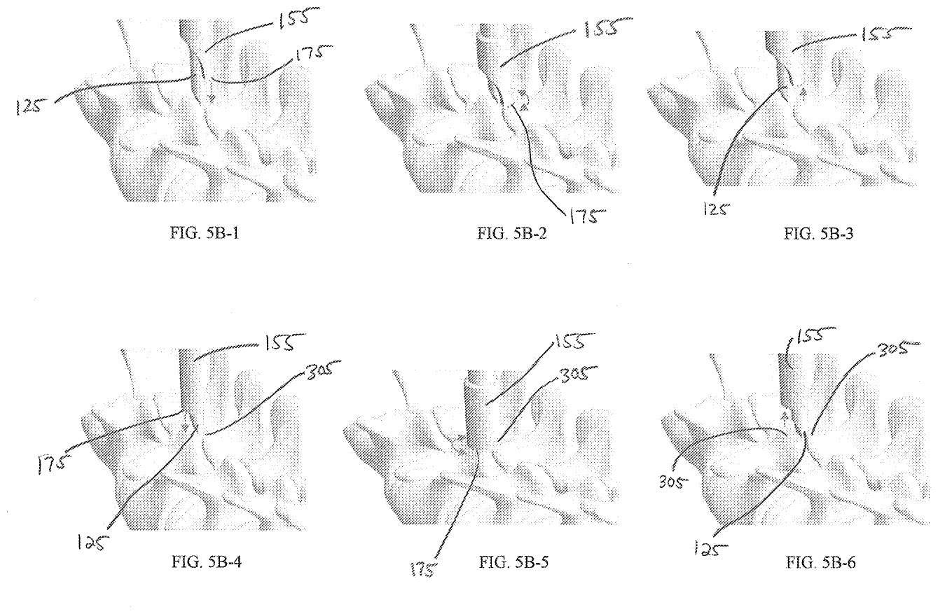

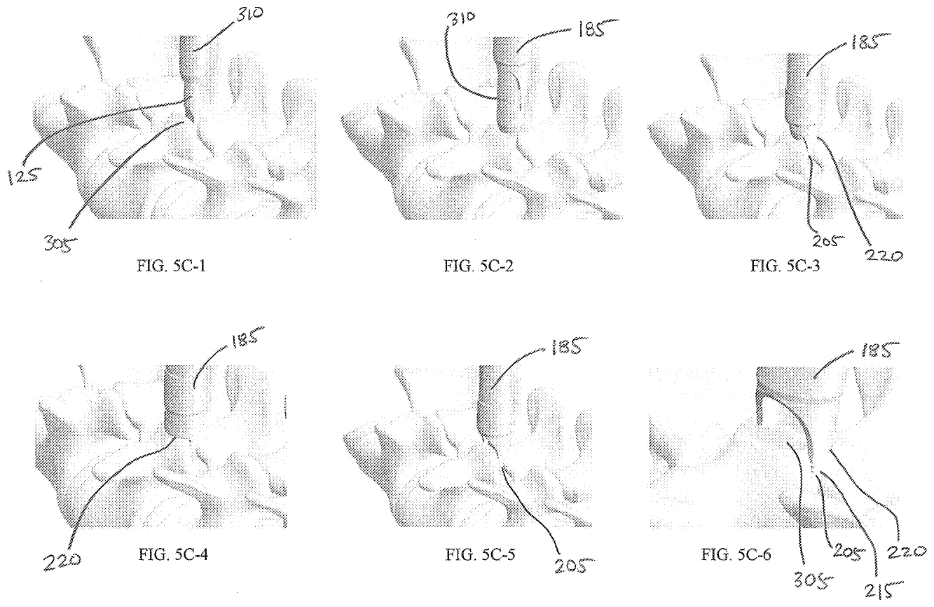

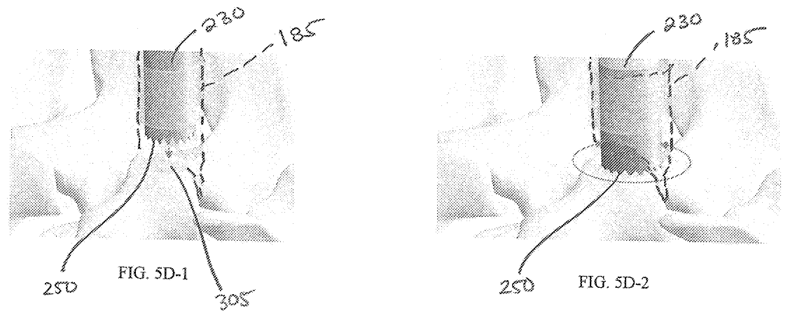

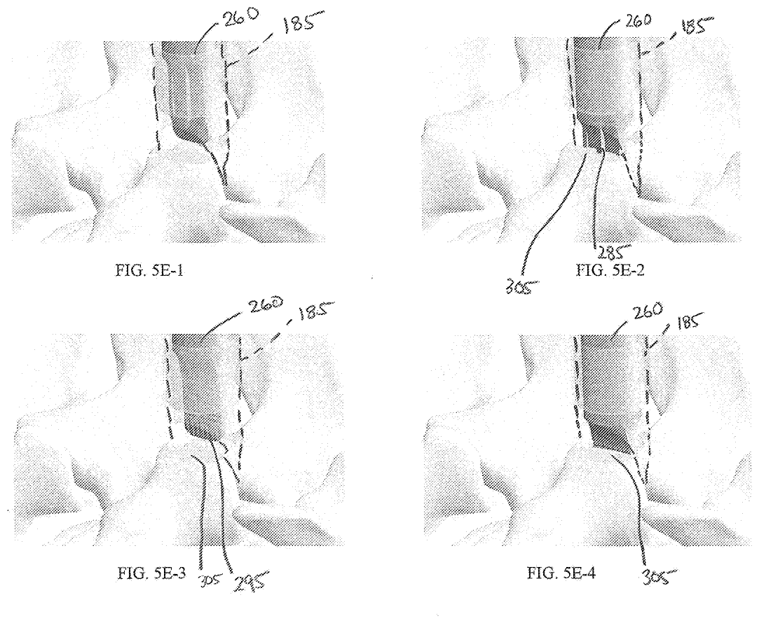

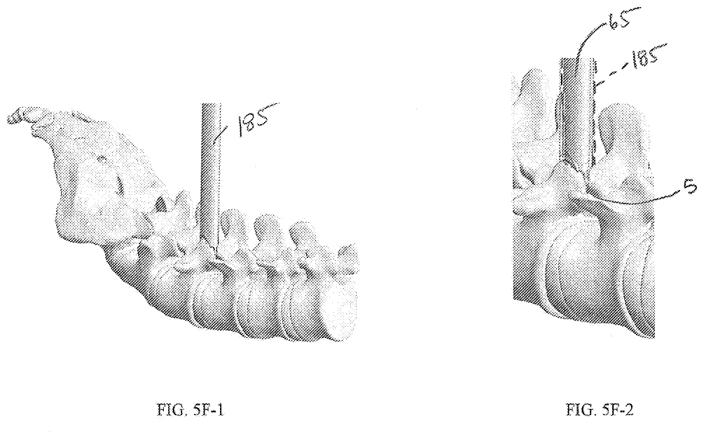

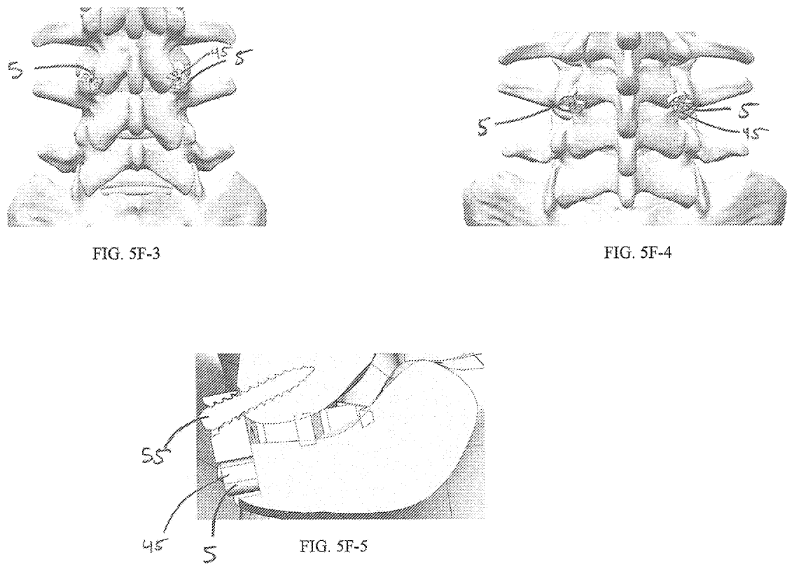

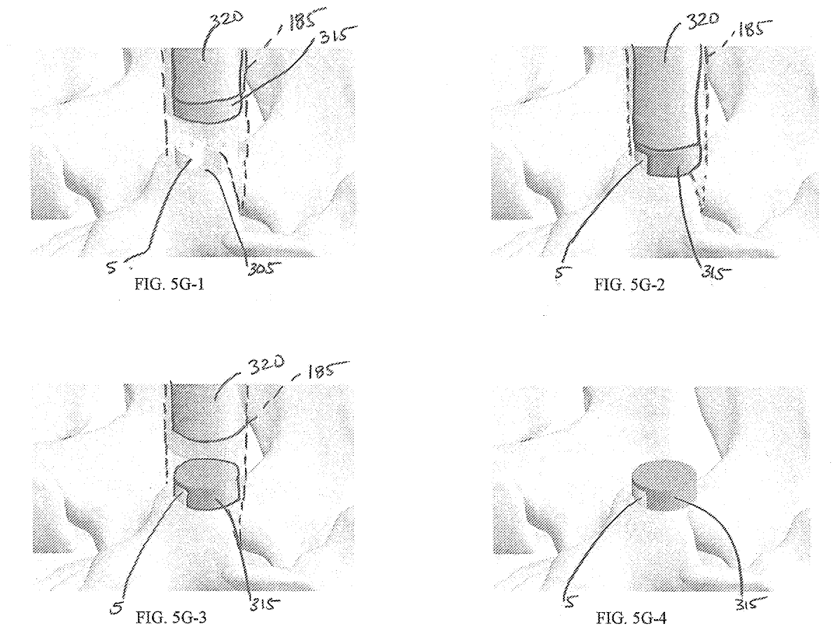

As shown in FIGS. 4B-1 to 4B-6, a first or outer decorticator 155 may include a tubular body 160 and a proximal portion 165 opposite a distal portion 170. The proximal portion 165 is adapted to receive a handle 167. The distal portion 170 may include bidirectional cutting teeth 175 in either a 180° pattern or array (see FIG. 4B-5) or a 360° pattern or array (see FIG. 4B-6). In the embodiment with the cutting teeth in a 180° pattern or array, there is also an arcuate cut-out feature 180 opposite the cutting teeth 175. The tubular body 160 defines a conduit with an inner diameter that is greater than the access chisel 125 such that the decorticator 155 can slide over the access chisel 125 during a procedure.

Turning now to FIGS. 4C-1 to 4C-4, the system may further include an access portal or guide tube 185. FIG. 4C-1 shows an isometric view of the access portal 185 having a tubular body 190 and a proximal portion 195 opposite a distal portion 200. The proximal portion 195 is adapted to receive a handle 197. The tubular body 190 defines a conduit 192 with an inner diameter that is greater than the access chisel 125 such that the access portal 185 can slide over the access chisel 125 during a procedure (see FIG. 4C-4). FIG. 4C-2 and -3 show an enlarged view of the distal portion 200. The distal portion 200 may include arms or forks 205 sized for the width of a lumbar facet joint. The arms or forks 205 form a tapered tip 210 which helps with ease of insertion into the joint. The arms or forks 205 also include teeth 215 to provide resistance to back out and side to side movement while using subsequent instruments. The distal portion 200 further includes a positive stop 220 opposite an arcuate shaped cut out 225. The positive stop 220 prevents over insertion into the joint and the cut out is contoured to the lumbar facet anatomy to prevent tissue interference during insertion.

As shown in FIGS. 4D-1 through 4D-6, the system further includes a second or intra-portal decorticator 230 which may include a solid tubular body 235 and a proximal portion 240 opposite a distal portion 245. The proximal portion 240 is adapted to receive a handle 242. FIG. 4D-2 and -3 show an enlarged view of the distal portion 245. The distal portion 245 may include bidirectional cutting teeth 250 in a 180° pattern or array (see FIG. 4D-4) or a 3600 pattern or array (see FIG. 4D-5) or a 360° pattern or array with a center countersink 252 (see FIG. 4D-6). In the embodiment with the cutting teeth 250 in a 180° pattern or array, there is also a linear (straight edge) cut-out feature 255 opposite the cutting teeth 250. In use, the decorticator 230 is received in the guide tube 185 during a procedure and is used to flatten bone and prepare the joint for fusion.

As shown in FIGS. 4E-1 through 4E-6, the system further includes a third or intra-facet decorticator 260 which may include a solid tubular body 265 and a proximal portion 270 opposite a distal portion 280. The proximal portion 270 is adapted to receive a handle 272. FIG. 4E-2 (side) and 4E-3 (isometric) and 4E-4 and -5 (top and bottom) are enlarged views of the distal portion 280. The distal portion 280 may include keel cutters 285 that are similar in design to those described above relative to the implant. The distal portion may also include a tapered tip 290 to ease insertion into the joint. Opposite the side with the keel cutters 285, the distal portion 280 further includes a pyramid tip rasp 295 design to roughen the articular surfaces of the lumbar facet. In use, the decorticator 260 is received in the guide tube 185 during a procedure and is used to remove tissue from the joint and prepare the articular surfaces for fusion.

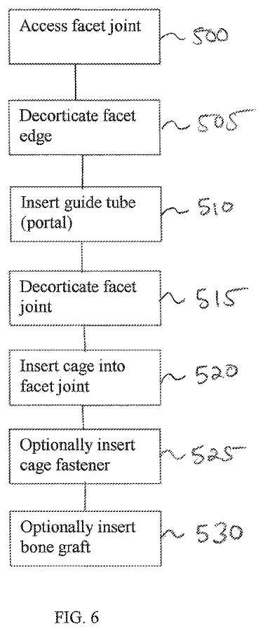

Turning now to FIGS. 5A-1 through FIG. 6, a method for lumbar fusion is shown and described. While the steps of the method are disclosed in a specific order, more or fewer steps may be used and the order may change. As shown in FIGS. 5A-1 through 5A-5 and described at step 500 of FIG. 6, the facet joint is accessed with an access chisel or access instrument 125. The thin, flat chisel portion of access chisel 125 enters the facet joint and the forked tip 140 stabilizes the chisel upon joint entrance (FIGS. 5A-1 to 5A-3) and helps to penetrate the facet capsule. As the chisel 125 is further inserted into the joint (see FIGS. 5A-4 and -5), the arcuate shaped cutout 150 prevents tissue interference during insertion.

As can be understood from FIGS. 5B-1 to 5B-6 and described at step 505 of FIG. 6, next, decortication of the facet edge with a trephine or the first or outer decorticator 155 that slides over the access chisel 125 is performed. As shown in FIG. 5B-1, the decorticator 155 slides over the access instrument 125 until the teeth 175 abut the medial facet surface. FIG. 5B-2 illustrates how the bidirectional teeth 175 remove cortical bone with pressure and a back and forth rotation. The user then slides the decorticator back up over the access instrument, as shown in FIG. 5B-3 and rotates the decorticator 180° and slides it back down the access chisel until the teeth 175 abut the lateral facet surface, as shown in FIG. 5B-4. FIG. 5B-5 illustrates how the bidirectional teeth 175 remove cortical bone with pressure and a back and forth rotation. This process is continued until a full 360° rotation can be achieved to ensure a flat bone surface 305, as shown in FIG. 5B-6, which shows the flat bone surface as the decorticator 155 is removed.

As can be understood from FIGS. 5C-1 to 5C-6 and described at step 510 of FIG. 6, the guide portal 185 is inserted over the access chisel 125 and the access chisel 125 is removed. As shown in FIG. 5C-1, an alignment tube 310 slides over the access instrument 125 until it abuts the facet entrance. The alignment tube 310 acts as a spacer between the access chisel 125 and the guide tube 185. The guide tube 185 slides over the alignment tube 310 in FIG. 5C-2. A snug fit guides the guide tube 185 to the joint with high precision. In FIG. 5C-3, the forks 205 are inserted into the joint until the positive stop 220 abuts the facet entrance on the medial side (superior facet). FIG. 5C-4 shows an alternative where the positive stop 220 can abut the lateral side of the facet joint entrance (inferior facet). FIG. 5C-5 shows the access chisel 125 and alignment tube being withdrawn from the guide tube 185 with minimal resistance. The teeth 215 on the forks 205 slightly distract the joint and anchor the position. FIG. 5C-6 is a close up or enlarged view of the guide tube 185 anchored into the lumbar facet joint after the access chisel 125 and the alignment tube 310 are removed.

Turning now to FIGS. 5D-1 and 2 and step 515 of FIG. 6, further decortication of the facet joint is performed and remodeling of bone in surrounding areas through the guide portal in preparation of cage implantation is performed. As shown in FIG. 5D-1, the second or intra-portal decorticator 230 is inserted through the guide tube 185 (shown as transparent) until the teeth contact the facet surface. FIG. 5D-2 illustrates the bidirectional cutting teeth 250 that allow for either back and forth or full rotational cutting in the clockwise or counter clockwise direction. The decorticator 230 can be removed through the guide tube 185 after decortication.

Turning to FIGS. 5E-1 to E4 and step 515 of FIG. 6, further decortication with a third or intra-facet decorticator 260 may be utilized. As shown in FIG. 5E-1, the decorticator 260 slides through the guide tube 185 (shown as transparent) to the entrance of the lumbar joint. In FIG. 5E-2, the decorticator 260 is inserted into the joint. Keels are cut (via keel 285) on one side of the joint and the rasp 295 removes cartilage and cortical bone on the opposite articulating surface. As shown in FIG. 5E-3, the decorticator 260 is removed from the joint and rotated 180°. Then, as shown in FIG. 5E-4, the decorticator 260 is re-inserted into the joint. This allows keels to be cut (via keel 285) and decortication to occur on both articulating surfaces. The decorticator is then removed from the joint.

Turning now to FIGS. 5F-1 and 5F-2, and step 520 of FIG. 6, the implant 5 is then introduced into the lumbar facet joint through the guide portal 185 by the delivery device 65 as described elsewhere herein. As shown in FIGS. 5F-3 to F5, and step 525 of FIG. 6, the screws 55 are placed into the implant 5 via the cage delivery instrument 65 to anchor the cage or implant 5 into the joint. FIGS. 5F3 and F4 show bilateral fixation of the facet joints at the treated level (L3/L4) and depict the lumbar facet cage or implant system in use in the lumbar spine. Fixation of the lumbar facet joints can be treated from L1 to S1 of the lumbar spine. The implant 5, when located in the facet joint space, provides lateral foraminal distraction and facet joint fixation. The wings 45 on the implant 5 act as hard stops to prevent over insertion into the joint space. The wings 45 further engage with a bone screw 55 for additional fixation.

Optionally, and as shown in FIGS. 5G-1 to 5G-4, and described at step 530 of FIG. 6, bone graft 315 is delivered into the surgical space through the guide portal and tamped down into the surrounding area. More specifically, FIG. 5G-1 shows a bone tamp 320 being used to deliver bone graft 315 down the guide tube 185 and to the back of the intra-facet implant 5 with high efficiency. FIG. 5G-2 shows the tamp 320 in use to press bone graft 315 over the back of the implant 5, filling in the gaps in and around the implant 5. As shown in FIG. 5G-3, the tamp 320 is removed from the guide tube 185, leaving the graft 315 with the implant 5. As shown in FIG. 5G-4, the guide tube 185 can then be removed and inserted on the other side for a bilateral placement or placed at another level to be treated and the implant 5 and bone graft 315 are left in place.

The above specification, examples and data provide a complete description of the structure and use of exemplary embodiments of the invention as defined in the claims. Although various embodiments of the claimed invention have been described above with a certain degree of particularity, or with reference to one or more individual embodiments, it is appreciated that numerous alterations to the disclosed embodiments without departing from the spirit or scope of the claimed invention may be possible. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

1. A lumbar facet implant comprising:
a proximal portion and a distal portion;
a first surface opposite a second surface, the first and second surface each including one or more teeth, each of the first and second surfaces extending between the proximal and distal portions; and
a stop feature extending from the proximal portion and generally perpendicular to one of the first surface or the second surface;
wherein the stop feature includes at least one opening configured to receive a fixation device.

2. The implant of claim 1, wherein the distal portion comprises a blunt distal tip.

3. The implant of claim 1, wherein the stop feature includes at least one rounded edge or corner.

4. The implant of claim 1, wherein the stop feature is a semi-circle in shape.

5. The implant of claim 4, wherein the stop feature extends from the first surface and includes two openings configured to receive a fixation device and the two openings are angled to extend above or away from the first surface.

6. The implant of claim 4, wherein the stop feature includes three openings configured to receive a fixation device and two openings are angled to extend above or away from the first surface and one opening is angled to extend below or away from the second surface.

7. The implant of claim 1, further comprising a second stop feature extending from the proximal portion and generally perpendicular to the other of the first or second surface.

8. The implant of claim 7, wherein the second stop feature comprises at least one rounded edge or corner.

9. The implant of claim 7, wherein the first and second stop features increase a height but not a width of the implant.

10.-13. (canceled)

14. A lumbar facet implant delivery system comprising:
a lumbar facet implant comprising a proximal portion and a distal portion, the proximal portion including a release driver opening and a fixation device opening; and
a delivery instrument comprising:
an elongated body comprising a distal end and a proximal end, the distal end defining a lumbar facet implant receiving feature that is complimentary in shape to the proximal portion of the lumbar facet implant;
a release driver configured to engage the release driver opening of the proximal portion of the implant; and
a tubular body defined within the elongated body that is configured to receive the fixation device and change the trajectory of the fixation device being delivered to the fixation device opening of the implant.

15. The system of claim 14, wherein the lumbar facet implant further comprises a stop feature extending from the proximal portion.

16.-20. (canceled)

21. A system for lumbar fusion comprising:
an access chisel;
at least one decorticator; and
a guide tube.

22. The system of claim 21, wherein the access chisel comprises a proximal end opposite a distal portion, the distal portion having a thin, forked, tapered tip, a positive stop and an arcuate shaped cut out opposite the positive stop.

23. The system of claim 21, wherein the at least one decorticator is a first decorticator comprising a tubular body and a proximal portion opposite a distal portion, the distal portion including bidirectional teeth in a 180° degree array or 360° degree array.

24. The system of claim 23, wherein the first decorticator has the bidirectional teeth in a 180° array and further includes an arcuate cut out feature opposite the bidirectional teeth.

25. (canceled)

26. The system of claim 21, wherein the at least one decorticator is a second decorticator comprising a tubular body and a proximal portion opposite a distal portion, the distal portion including bidirectional teeth in a 180° degree array, a 360° degree array or a 360° degree array with a center countersink.

27. The system of claim 26, wherein the second decorticator has the bidirectional teeth in a 180° array and further includes a linear or straight edge cut out feature opposite the bidirectional teeth.

28. (canceled)

29. The system of claim 21, wherein the at least one decorticator is a third decorticator comprising a solid tubular body and a proximal portion opposite a distal portion, the distal portion including one side having a keel and a second side having a rasp.

30. (canceled)

31. The system of claim 21, wherein the at least one decorticator comprises a first decorticator, a second decorticator and a third decorticator.

32. (canceled)

33. (canceled)

34. The system of claim 31, wherein the first decorticator is adapted to slide over the access chisel, the second decorticator is received in the guide tube, and the third decorticator is received in the guide tube.
